(19) United States
(12) Patent Application Publication
(10) Pub. No.: US 2021/0259965 A1
(43) Pub. Date: Aug. 26, 2021

(54) ANTIVIRAL PREPARATION FOR TREATING HUMAN SKIN

(71) Applicant: Michael K. Hoskins, Carl Junction, MO (US)

(72) Inventor: Michael K. Hoskins, Carl Junction, MO (US)

(21) Appl. No.: 17/302,428

(22) Filed: May 3, 2021

Related U.S. Application Data
(62) Division of application No. 16/800,069, filed on Feb. 25, 2020.

(51) Int. Cl. A61K 9/06; A61K 31/191; A61K 47/06; A61K 33/20

(52) U.S. Cl. CPC A61K 9/06; A61K 33/20; A61K 47/06; A61K 31/191

(57) ABSTRACT

An antiviral preparation for treating skin includes an alpha hydroxy acid and a source of chlorine dioxide in a waterless ointment base. This antiviral preparation can be manufactured at a low cost, and is very effective in treating topical viral infections, such as cold sores, warts, genital herpes, shingles, and molluscum contagiosum. The antiviral preparation substantially reduces the healing time for cold sores. In addition, one formulation of the antiviral preparation is suitable as a pour-on treatment for burns.

### BACKGROUND

### 1. Technical Field

This disclosure generally relates to preparations for treating human skin, and more specifically relates to antiviral preparations for treating human skin.

### 2. Background Art

Over time, many preparations for treating human skin have been developed and used, some with varying success. Some of these have antiviral properties and can be used to treat skin conditions caused by viruses, such as cold sores, genital herpes, shingles, and molluscum contagiosum. Zovirax, a brand name for acyclovir that is manufactured by GlaxoSmithKline, comes in an ointment and a cream that can be used to treat cold sores and genital herpes. Zovirax is a registered trademark of GlaxoSmithKline.

Many different preparations for treating cold sores have been developed and marketed. Many of these preparations have a very small benefit at a very high cost. For example, Abreva, a docosanol cream that is manufactured by GlaxoSmithKline, is marketed by stating a person can get rid of a cold sore in as few as 2.5 days when used at the first sign of a cold sore, with 4.1 days being the median time for getting rid of a cold sore. Abreva is a registered trademark of GlaxoSmithKline. It is not clear what constitutes “getting rid of” a cold sore according to the marketing materials for Abreva. For many people, a cold sore has a life of about two weeks from the first tingling to getting rid of the cold sore. Many people who use Abreva have their normal time cycle for a cold sore reduced by one to three days. Abreva costs over $15 for a 2 gram tube, making this cream very expensive. Other known cold sore remedies suffer from similar problems, only marginally reducing the time for having a cold sore at a very high cost.

### BRIEF SUMMARY

An antiviral preparation for treating skin includes an alpha hydroxy acid and a source of chlorine dioxide in a waterless ointment base. This antiviral preparation can be manufactured at a low cost, and is very effective in treating topical viral infections, such as cold sores, warts, genital herpes, shingles, and molluscum contagiosum. The antiviral preparation substantially reduces the healing time for cold sores. In addition, one formulation of the antiviral preparation is suitable as a pour-on treatment for burns.

The foregoing and other features and advantages will be apparent from the following more particular description, as illustrated in the accompanying drawings.

### BRIEF DESCRIPTION OF THE SEVERAL VIEWS OF THE DRAWING(S)

The disclosure will be described in conjunction with the appended drawings, where like designations denote like elements, and:

FIG. 1 shows preferred ingredients for the antiviral preparation disclosed and claimed herein;

FIG. 2 shows a preferred formulation for a first embodiment of the antiviral preparation;

FIG. 3 shows a preferred formulation for a second embodiment of the antiviral preparation;

FIG. 4 shows a preferred formulation for a third embodiment of the antiviral preparation;

FIG. 5 shows a preferred formulation for a fourth embodiment of the antiviral preparation;

FIG. 6 shows a preferred formulation for a fifth embodiment of the antiviral preparation;

FIG. 7 is a table that shows melting points for different alpha hydroxy acids;

FIG. 8 is a flow diagram of a method for making the antiviral preparation; and

FIG. 9 is a flow diagram of a specific method within the scope of the method in FIG. 8 for making the antiviral preparation.

### DETAILED DESCRIPTION

As discussed in the Background Art section above, known antiviral preparations for treating skin have limited effectiveness and high cost. An antiviral preparation for treating skin as disclosed and claimed herein includes an alpha hydroxy acid and a source of chlorine dioxide in a waterless ointment base. This antiviral preparation can be manufactured at a low cost, and is very effective in treating topical viral infections, such as cold sores, warts, genital herpes, shingles, and molluscum contagiosum. The antiviral preparation substantially reduces the healing time for cold sores. In addition, one formulation of the antiviral preparation is suitable as a pour-on treatment for burns.

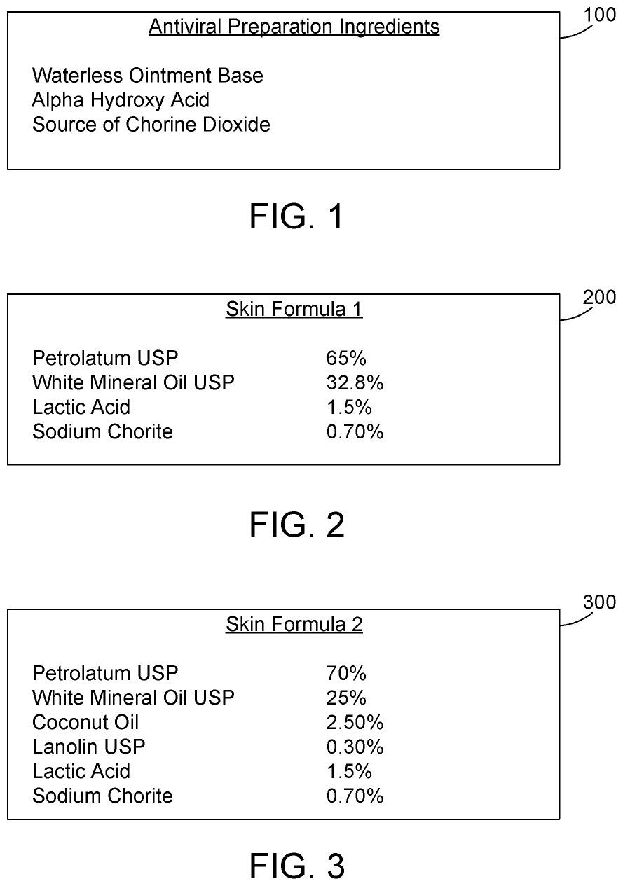

As shown in FIG. 1, the antiviral preparation disclosed and claimed herein preferably has ingredients shown at 100 that include: waterless ointment base; alpha hydroxy acid; and a source of chlorine dioxide. Suitable waterless ointment bases preferably include any waterless ointment base that is solid or semisolid at 20-39° C., melts at or near 37° C., and is miscible with some alpha hydroxy acids at temperatures greater than 50° C. Suitable alpha hydroxy acids include, without limitation, glycolic acid, lactic acid, malic acid, citric acid, and tartaric acid. Lactic acid is the most preferred of the alpha hydroxy acids due to its lower melting point. However, any suitable alpha hydroxy acid could be used. Suitable sources of chlorine dioxide include gaseous chlorine dioxide from a chlorine dioxide generator, or any suitable granules or powder such as alkali earth metal chlorite or chlorate, two suitable examples of which are NaClO₂ and NaClO₃.

The ingredients in FIG. 1 can be formulated in suitable proportions to arrive at different formulations for the antiviral preparation. Note the term “USP” as used herein stands for “United States Pharmacopoeia”, which has published standards for many ingredients. Thus, petrolatum USP is petrolatum that complies with the specifications published by USP. In a first formulation for treating skin according to a first embodiment of the antiviral preparation, the antiviral preparation comprises petrolatum USP, white mineral oil USP, lactic acid, and sodium chlorite. Note the proportions herein are designated in percentages by weight (w/w).

The proportions of these ingredients are preferably:

| | |
|---|---|
| Petrolatum USP | 10-98% |
| White Mineral Oil USP | 10-90% |
| Lactic Acid | 1.0-5.0% |
| Sodium Chlorite (NaClO²) | 0.50-3.0% |

The proportions of these ingredients are more preferably:

| | |
|---|---|
| Petrolatum USP | 30-90% |
| White Mineral Oil USP | 9.0-69% |
| Lactic Acid | 1.5-2.0% |
| Sodium Chlorite (NaClO²) | 0.50-2.0% |

The proportions of these ingredients are most preferably:

| | |
|---|---|
| Petrolatum USP | 65% |
| White Mineral Oil USP | 32.8% |
| Lactic Acid | 1.5% |
| Sodium Chlorite (NaClO²) | 0.70% |

The most preferred proportions for the first embodiment of the antiviral preparation are shown at 200 in FIG. 2.

In a second formulation for treating skin according to a second embodiment of the antiviral preparation, the antiviral preparation comprises petrolatum USP, white mineral oil USP, coconut oil, lanolin USP, lactic acid, and sodium chlorite. The proportions of these ingredients are preferably:

| | |
|---|---|
| Petrolatum USP | 10-98% |
| White Mineral Oil USP | 10-90% |
| Coconut Oil | 0.5-50% |
| Lanolin | 0.5-3.0% |
| Lactic Acid | 1.0-5.0% |
| Sodium Chlorite (NaClO²) | 0.20-3.0% |

The proportions of these ingredients are more preferably:

| | |
|---|---|
| Petrolatum USP | 50-88.5% |
| White Mineral Oil USP | 15-50% |
| Coconut Oil | 1.0-7.0% |
| Lanolin | 0.1-1.5% |
| Lactic Acid | 1.5-2.0% |
| Sodium Chlorite (NaClO²) | 0.50-2.0% |

The proportions of these ingredients are most preferably:

| | |
|---|---|
| Petrolatum USP | 70% |
| White Mineral Oil USP | 25% |
| Coconut Oil | 2.50% |
| Lanolin | 0.30% |
| Lactic Acid | 1.5% |
| Sodium Chlorite (NaClO²) | 0.70% |

The most preferred proportions for the second embodiment of the antiviral preparation are shown at 300 in FIG. 3. The addition of coconut oil and lanolin in the formulation of the second embodiment provides skin conditioning ingredients that help to soothe and heal skin.

One of the principal concerns when treating second or third degree burns is the risk of infection. The risk of infection can be significantly reduced using the antiviral preparation disclosed herein, which also has antibacterial properties. In the treatment of burns, a formulation that is thin enough to pour on a burn injury wound would be preferable to a formulation that must be rubbed on the burn injury, which would cause pain to the patient. A third formulation for treating skin according to a third embodiment of the antiviral preparation is pourable so a burn injury can be treated by pouring on the antiviral preparation without rubbing or otherwise agitating the burn injury. This third formulation would have particular utility in hospital emergency rooms and burn centers. In the third formulation of the antiviral preparation according to the third embodiment, the antiviral preparation comprises petrolatum USP, white mineral oil USP, lactic acid, and sodium chlorite. The proportions of these ingredients are preferably:

| | |
|---|---|
| Petrolatum USP | 10-30% |
| White Mineral Oil USP | 10-90% |
| Lactic Acid | 1.0-3.0% |
| Sodium Chlorite (NaClO²) | 0.10-3.0% |

The proportions of these ingredients are more preferably:

| | |
|---|---|
| Petrolatum USP | 15-25% |
| White Mineral Oil USP | 75-80% |
| Lactic Acid | 1.5-2.0% |
| Sodium Chlorite (NaClO²) | 0.50-1.5% |

The proportions of these ingredients are most preferably:

| | |
|---|---|
| Petrolatum USP | 19.8% |
| White Mineral Oil USP | 78% |
| Lactic Acid | 1.5% |
| Sodium Chlorite (NaClO²) | 0.70% |

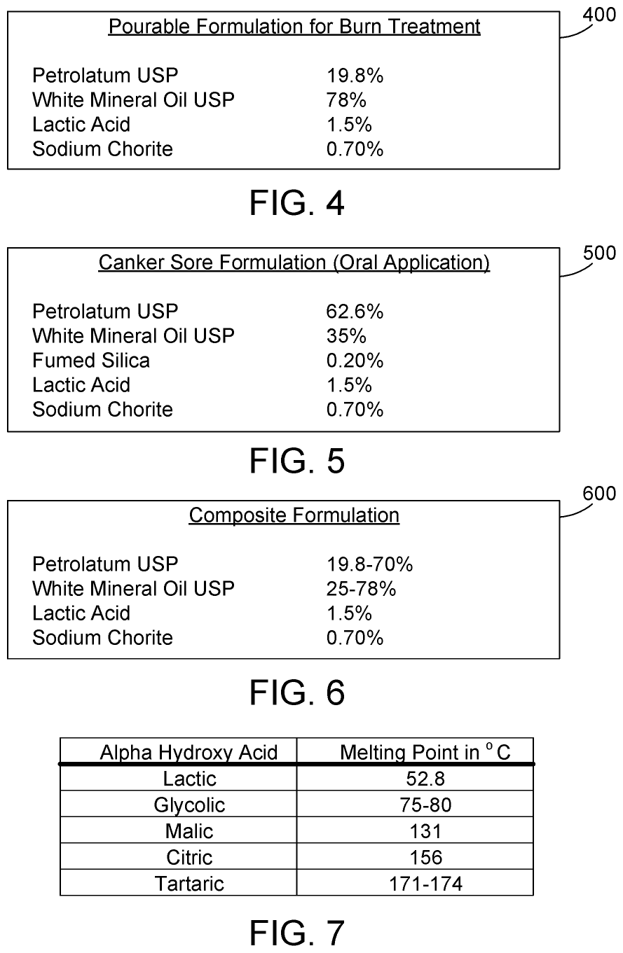

The most preferred proportions for the third embodiment of the antiviral preparation are shown at 400 in FIG. 4. By providing a pourable formulation, the antiviral preparation can be poured onto a burn or other wound without having to rub or otherwise irritate the wound.

A fourth formulation for treating skin according to a fourth embodiment of the antiviral preparation can be used as an oral application in a person's mouth, such as in treating canker sores. In the fourth formulation of the antiviral preparation according to the fourth embodiment, the antiviral preparation comprises petrolatum USP, white mineral oil USP, fumed silica, lactic acid, and sodium chlorite. The fumed silica serves as a micro abrasive to remove the outer mucoid layer of the canker sore. The proportions of these ingredients are preferably:

| | |
|---|---|
| Petrolatum USP | 20-98% |
| White Mineral Oil USP | 10-80% |
| Fumed Silica | 0.10-1.0% |
| Lactic Acid | 1.0-5.0% |
| Sodium Chlorite (NaClO²) | 0.50-3.0% |

The proportions of these ingredients are more preferably:

| | |
|---|---|
| Petrolatum USP | 30-80% |
| White Mineral Oil USP | 20-70% |
| Fumed Silica | 0.20-0.50% |
| Lactic Acid | 1.5-2.0% |
| Sodium Chlorite (NaClO²) | 0.50-2.0% |

The proportions of these ingredients are most preferably:

| | |
|---|---|
| Petrolatum USP | 62.6% |
| White Mineral Oil USP | 35% |
| Fumed Silica | 0.20% |
| Lactic Acid | 1.5% |
| Sodium Chlorite (NaClO²) | 0.70% |

The most preferred proportions for the fourth embodiment of the antiviral preparation are shown at 500 in FIG. 5. Although canker sores are not of viral origins, the preparation of the fourth embodiment is well-suited to treating canker sores because it is an anti-inflammatory that works to reduce the inflammation. In addition, the preparation has a keratolytic effect that helps to heal the canker sore.

If the ranges of the four embodiments disclosed in detail above are taken together, a composite formulation for a fifth embodiment of the antiviral preparation can be derived by taking the low and high numbers from all the previous four formulations. For the fifth embodiment, the proportions of the common ingredients are preferably:

| | |
|---|---|
| Petrolatum USP | 10-98% |
| White Mineral Oil USP | 10-90% |
| Lactic Acid | 1.0-5.0% |
| Sodium Chlorite (NaClO²) | 0.50-3.0% |

The proportions of these ingredients are more preferably:

| | |
|---|---|
| Petrolatum USP | 15-90% |
| White Mineral Oil USP | 9.0-80% |
| Lactic Acid | 1.5-2.0% |
| Sodium Chlorite (NaClO²) | 0.50-2.0% |

The proportions of these ingredients are most preferably:

| | |
|---|---|
| Petrolatum USP | 19.8-70% |
| White Mineral Oil USP | 25-78% |
| Lactic Acid | 1.5% |
| Sodium Chlorite (NaClO²) | 0.70% |

The most preferred proportions for the composite formulation of the antiviral preparation are shown at 600 in FIG. 6.

The proportions of ingredients can be varied within the ranges shown above to obtain different melting points for the antiviral preparation. The melting point for the antiviral preparation is most preferably 32-43° C., which allows the antiviral preparation to melt or partially melt when put on human skin.

All of the five specific embodiments disclosed above include both lactic acid and sodium chlorite, which are both known antibacterials. The preparations above are therefore not only antiviral, but antibacterial as well.

While specific ingredients are shown in the formulations above, suitable substitutions can be made within the scope of the disclosure and claims herein. For example, any of the following including a suitable combination could be substituted for a portion of the petrolatum: almond butter, aloe butter, refined avocado butter, refined cocoa butter, coconut butter, olive butter, refined shea butter, refined soy butter, cross-linked dimethicone, and palm oil. Silicon oil (dimethicone) could be used instead of mineral oil in all of the formulations above. Other oils could also be used, including without limitation castor oil, cottonseed oil, linseed oil, olive oil, palm kernel oil, peanut oil, rapeseed oil, sunflower oil, soybean oil, and tung oil. Any suitable alpha hydroxy acid could be used. These acids have different melting points, and thus can be selected and their proportions varied to achieve a desired melting point for the antiviral preparation. The melting points for five different alpha hydroxy acids are shown in FIG. 7. Lactic acid and glycolic acid are most preferred due to their lower melting points and due to their keratolytic effect on human skin. Other sources for chlorine dioxide besides sodium chlorite could also be used, including gaseous chlorine dioxide from a chlorine dioxide generator and sodium chlorate (NaClO₃).

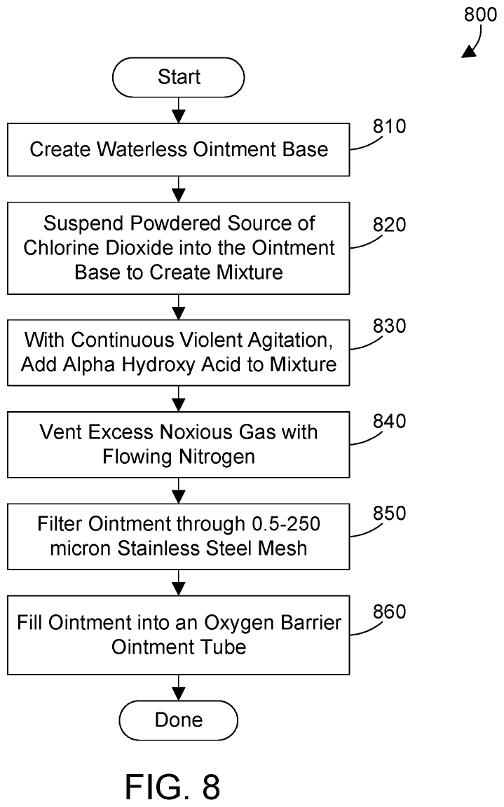

FIG. 8 shows one suitable method 800 for making the antiviral preparation in accordance with the disclosure and claims herein. A waterless ointment base is created (step 810). One suitable example of a waterless ointment base is a mixture of petrolatum USP and white mineral oil USP, as shown in the specific formulations above. However, any suitable waterless ointment base could be used. A powdered source of chlorine dioxide is then suspended into the ointment base to create a mixture (step 820). One suitable example of a powdered source of chlorine dioxide is powdered sodium chlorite. With continuous violent agitation, an alpha hydroxy acid is added to the mixture (step 830). One suitable mixer that provides the violent agitation needed in step 830 is a Robot Coupe immersion blender, which is a commercial immersion blender often marketed to restaurants. Excess noxious gas is vented with flowing nitrogen (step 840). The ointment is then filtered through a 0.5-250 micron stainless steel mesh (step 850). The filtering in step 850 removes unreacted sodium chlorite and reacted sodium chloride. Using a finer mesh filter slows the filtering process, while using a coarser mesh can speed up the filtering process considerably. In practice, 100 micron stainless steel mesh works well to remove most of the unreacted sodium chlorite and reacted sodium chloride while providing a reasonable filtering time. The ointment is then filled into an oxygen barrier ointment tube (step 860). Method 800 is then done.

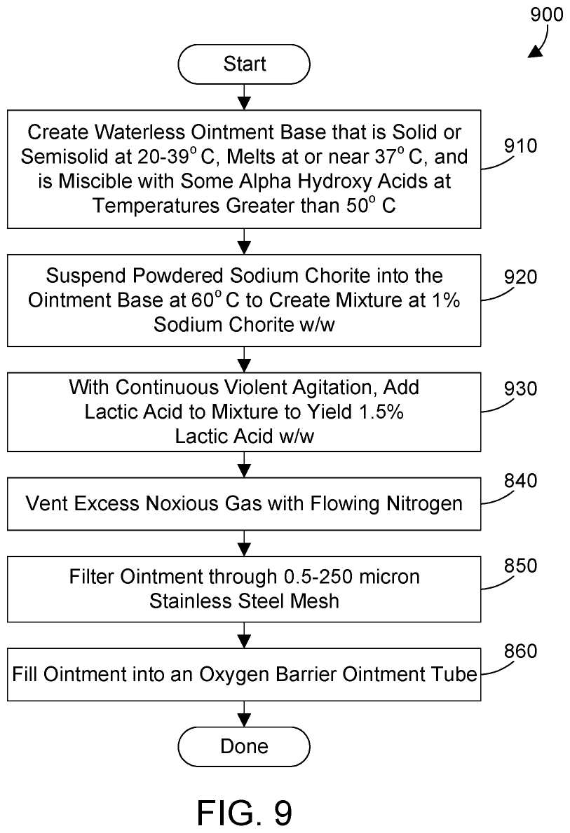

One specific method that is within the scope of method 800 in FIG. 8 is shown as method 900 in FIG. 9. Referring to FIG. 9, method 900 begins by creating a waterless ointment base that is solid or semisolid at 20-39° C., melts at or near 37° C., and is miscible with some alpha hydroxy acids at temperatures greater than 50° C. (step 910). Powdered sodium chlorite is then suspended into the ointment base at 60° C. to create a mixture at 1% sodium chlorite w/w (step 920). With continuous violent agitation, lactic acid is added to the mixture to yield 1.5% lactic acid w/w (step 930). The addition of lactic acid in step 930 can be performed in any suitable way. In a first example, a powdered form of 100% lactic acid could be melted into the mixture at 60° C. in step 930. In a second example, 100% lactic acid mixed with water to provide a 90% lactic acid liquid could be slowly added to the mixture in step 930. Note that 90% lactic acid liquid is a common raw material used in food and pharmaceuticals. Of course, other methods could be used to add lactic acid to the mixture in step 930 within the scope of the disclosure and claims herein. Steps 840, 850 and 860 are then performed, which were discussed above with reference to FIG. 8. Method 900 is then done.

Method 900 in FIG. 9 was developed by the inventors after considerable testing. For example, mixing sodium chlorite and acid at temperatures of 50-100° C. can produce an explosive reaction. The steps in method 900 are the product of significant research, development, and experimentation, so the specific combination of steps in method 900 would not have been obvious to one of ordinary skill in the art.

The antiviral preparation disclosed herein has other unexpected benefits in addition to being antiviral. The antiviral preparation has antibiotic properties, as well as anti-inflammatory properties that cause the preparation be a topical analgesic and anesthetic as well.

The specific ingredients and proportions above can be generalized. For example, the antiviral preparation could include any suitable waterless ointment base in any suitable proportion, any suitable alpha hydroxy acid in any suitable proportion, and any source of chlorine dioxide in any suitable proportion. The disclosure and claims herein expressly extend to any suitable antiviral preparation that includes ingredients in ranges disclosed herein.

The antiviral preparation disclosed and claimed herein is extremely effective in killing both bacteria and viruses, and can be manufactured at low cost. The result is an antiviral preparation that is much less expensive to manufacture and use.

The benefits of using sodium chlorite and of using various acids to treat human skin is known in the art. For example, the following U.S. patents describe the therapeutic benefits of using sodium chlorite and/or an acid: U.S. Pat. Nos. 4,330,531; 5,384,134; RE37,263; 5,516,799; and 8,784,901. Each of these patents disclose ingredients in an aqueous medium. The embodiments disclosed and claimed herein include a waterless ointment base. The use of a waterless ointment base allows the sodium chlorite and the alpha hydroxy acid to remain suspended in the waterless ointment base until the ointment is placed on human skin, at which time the moisture in the skin will activate all of the beneficial effects of the sodium chlorite and alpha hydroxy acid.

An antiviral preparation for treating skin includes an alpha hydroxy acid and a source of chlorine dioxide in a waterless ointment base. This antiviral preparation can be manufactured at a low cost, and is very effective in treating topical viral infections, such as cold sores, warts, genital herpes, shingles, and molluscum contagiosum. The antiviral preparation substantially reduces the healing time for cold sores. In addition, one formulation of the antiviral preparation is suitable as a pour-on treatment for burns.

One skilled in the art will appreciate that many variations are possible within the scope of the claims. Thus, while the disclosure is particularly shown and described above, it will be understood by those skilled in the art that these and other changes in form and details may be made therein without departing from the spirit and scope of the claims.

1. A method for manufacturing a preparation for treating human skin, the method comprising:
creating a waterless ointment base;
suspending a powdered source of chlorine dioxide into the ointment base to create a mixture; and
with continuous violent agitation, adding an alpha hydroxy acid to the mixture.

2. The method of claim 1 further comprising:
venting excess noxious gas with flowing nitrogen;
filtering the preparation through a 0.5-250 micron stainless steel mesh; and
filling an oxygen barrier ointment tube with the preparation.

3. The method of claim 1 wherein the waterless ointment base is solid or semisolid at 20-39° C., melts near 37° C., and is miscible with some alpha hydroxy acids at temperatures greater than 50° C.

4. The method of claim 1 wherein the waterless ointment base comprises petrolatum.

5. The method of claim 4 wherein the waterless ointment base further comprises mineral oil.

6. The method of claim 1 wherein the waterless ointment base comprises petrolatum that is 98% of the preparation w/w.

7. The method of claim 5 wherein the waterless ointment base comprises petrolatum that is petrolatum that is 10-90% of the preparation w/w and mineral oil that is 10-90% of the preparation w/w.

8. The method of claim 1 wherein the source of chlorine dioxide comprises sodium chlorite that is 0.50-3.0% of the preparation w/w.

9. The method of claim 1 wherein the alpha hydroxy acid comprises lactic acid that is 1.0-5.0% of the preparation w/w.

10. The method of claim 1 wherein:
the waterless ointment base comprises 10-90% petrolatum w/w and 10-90% mineral oil w/w;
the source of chlorine dioxide comprises 0.50-3.0% sodium chlorite w/w; and
the alpha hydroxy acid comprises 1.0-5.0% lactic acid w/w.

11. A method for manufacturing a preparation for treating human skin, the method comprising:
creating a waterless ointment base that is solid or semisolid at 20-39° C., melts near 37° C., and is miscible with some alpha hydroxy acids at temperatures greater than 50° C.;
suspending powdered sodium chlorite into the ointment base at 60° C. to create a mixture at 1.0% sodium chlorite w/w;
with continuous violent agitation, adding lactic acid to the mixture to yield 1.5% lactic acid w/w;
venting excess noxious gas with flowing nitrogen;
filtering the preparation through a 0.5-250 micron stainless steel mesh; and
filling an oxygen barrier ointment tube with the preparation.

12. The method of claim 1 further comprising filtering the preparation through a stainless steel mesh.

13. The method of claim 12 further comprising filling an oxygen barrier ointment tube with the preparation.

14. A method for manufacturing a preparation for treating human skin, the method comprising:
creating a waterless ointment base;
suspending a powdered source of chlorine dioxide into the ointment base to create a mixture; and
with continuous violent agitation, adding an alpha hydroxy acid to the mixture;
filtering the preparation through a 0.5-250 micron stainless steel mesh; and
filling an oxygen barrier ointment tube with the preparation.
